(19) United States
(12) Patent Application Publication
(10) Pub. No.: US 2008/0003132 A1
(43) Pub. Date: Jan. 3, 2008

(54) Volatile Liquid Disseminator

(75) Inventors: Thomas McGee, Nyack, NY (US); Colin Brown, Berkshire (GB); Guy Edward Naish, Oxfordshire (GB)

Correspondence Address: NORRIS, MCLAUGHLIN & MARCUS, 875 THIRD AVE, 18TH FLOOR, NEW YORK, NY, 10022, US

(73) Assignee: GIVAUDAN SA, Vernier (CH)

(21) Appl. No.: 10/597,533

(22) PCT Filed: Feb. 22, 2005
(86) PCT No.: PCT/CH05/00101

§ 371 Date: Oct. 24, 2006

Related U.S. Application Data
(60) Provisional application No. 60/547,119, filed on Feb. 24, 2004.

(51) Int. Cl. A61L 9/04; A61L 9/12; B01D 53/86

(52) U.S. Cl. 422/4; 422/122

(57) ABSTRACT

An apparatus adapted to both purify an air flow passing through it and disseminate a volatile liquid therein, comprising a source of volatile liquid, a disseminating element for the liquid and a catalyst adapted to remove pollutants at relatively low temperatures, the aforementioned elements being arranged such that the air flow encounters first the catalyst and then the disseminating element. The apparatus is useful, for example, for purifying air and adding fragrance thereto.

This invention relates to devices for disseminating volatile liquids into an atmosphere, and more especially to such devices useful for purifying such atmospheres.

Devices for disseminating volatile liquids in vapour form into an atmosphere are well known. Such liquids include fragrances, insecticides, antifungal and anti-mould compositions and medicaments. The variety of devices available is wide and includes fan-powered devices, this having been made possible by the availability of relatively cheap fans and blowers, arising from the computer industry.

Air purifiers, which clean the air of pollutants and by passing it through a cleansing element, such as a filter or an absorbent material, are also well known.

It has now been found that it is possible to provide a single device that conveniently combines the functions of air purifier and volatile liquid disseminator. The invention therefore provides an apparatus adapted to both purify an air flow passing through it and disseminate a volatile liquid therein, comprising a source of volatile liquid, a disseminating element for the liquid and a catalyst adapted to remove pollutants at relatively low temperatures, the aforementioned elements being arranged such that the air flow encounters first the catalyst and then the disseminating element.

The invention also provides a method of simultaneously purifying and disseminating into an atmosphere a volatile liquid, comprising causing the atmosphere to pass in order over a catalyst adapted to remove pollutants at relatively low temperatures and a volatile liquid disseminating element.

The air flow may be achieved by any desired means. For example, it may be a means of forced air circulation, such as a fan or a blower, which is powered by electricity from the mains, generator, battery or solar cells. In the case of mains electricity, the apparatus may include a suitable transformer or rectifier. This form of air circulation is useful if the invention is to be used as part of a self-contained apparatus for use, for example, in domestic situations. However, it may also be an air flow generated outside the apparatus, for example, an air flow produced by a remote fan and then conveyed through a duct, such as in a ventilation system, or the air flow produced in the ventilation system of a motor vehicle by its movement or by means of a fan or a combination of these.

The source of volatile liquid may be any suitable reservoir of any suitable material. The disseminating element may be any such element suitable for disseminating volatile liquid in vapour form into an atmosphere when exposed to an air current. There are many possibilities that are encompassed by this invention, but the preferred element is a porous wick. Wicks are well known to the art and they can be made of any suitable material, such as compressed fibres, porous plastics, graphite and ceramics. Examples of especially suitable materials are the porous polyethylene and polypropylene wicks obtained from manufacturers such as Porex or Micropore.

The wick has one end immersed in the volatile liquid in a reservoir and the other exposed to the air current. Liquid is drawn into the wick by capillary action and the evaporation of the liquid from the end exposed to the air current causes more to be drawn from the reservoir. The reservoir is preferably part of a replaceable refill, which will be considered in greater detail hereinunder.

The function of the catalyst is to remove pollutants from the atmosphere. Any suitable catalyst can be used to do this. The catalyst is typically formed on a monolith, an open-pored inert support such as ceramic or aluminium. This structure allows the free passage of air through the catalyst. Such monoliths are commonly used in automotive catalytic converters. On to the support is deposited a suitable catalyst which is formed of a precious metal with a metal oxide. As temperatures in an apparatus for use in an enclosed atmosphere, such as a room, cannot be too high, from the points of view both of safety and of expense of construction to withstand high temperatures, the catalyst is preferably one that functions at room temperature or at the temperature levels usually encountered in domestic appliances (approximately 60-70° C. for electrical air fresheners, and 100-140° C. for electrical insecticide dispensers).

Suitable catalysts include those made from platinum and palladium, but a gold-containing catalyst is preferred, as such catalysts are known to function at relatively low temperatures, even ambient temperature. Mixed alloy systems (e.g. Au/Pt) may be used. The preferred metal oxide is iron oxide but other metal oxides and blends thereof may be used. The catalyst composition is formulated depending on the target gases to be catalysed and one skilled in the art would be able to prepare such blends. Given the cost of precious metals, the amount of catalyst present is not normally more than 1.0 g of a 5% weiglht mixture of precious metal in metal oxide.

Where necessary, the support may also comprise heating elements, which raise the temperature of the catalyst to its correct operational range. The preferred arrangement is a metal monolith comprising the catalyst, with a flexible printed resistor wrapped around it. In the case of a domestic air purification/dissemination appliance, the heating elements may derive their electricity from the same source as does the means of forced air circulation. The catalyst, with or without heating elements, maybe designed to be removed easily from the apparatus for regeneration or replacement. In the case of an apparatus in which the source of air flow is remote, any suitable electricity supply may be arranged.

The arrangement of the elements of the apparatus according to the invention are such that the air flow passes first the catalyst and then the volatile liquid disseminator Any suitable arrangement that gives this result may be used. In a typical arrangement in a domestic appliance, a fan is positioned between the catalyst and the disseminating element, such that it draws air over the catalyst and blows this air over the disseminating element. The elements are mounted so that this is achieved, for example, in a cylindrical duct.

Other elements can also be utilized in this invention. For example, it is preferred to mount in the path of the airflow prior to its arrival at the catalyst a filter for the removal of dust and other particulate matter. This makes the catalyst more efficient and gives longer catalyst life, an important consideration when expensive materials such as gold are involved. A typical dust filter comprises a non-woven fabric that does not significantly impair airflow but that entraps dust, pollen and other airborne particulate substances. The filter may be given enhanced particulate trapping characteristics, for example, by imparting thereto an electrostatic charge or coating with a tackifying agent.

The particulate filter may be augmented by a filter for the removal of materials associated with malodour. Such a filter, which should also have an open structure in order not to restrict air flow to any significant degree, is placed intermediate between the particulate filter and the volatile liquid disseminator and is typically activated charcoal or zeolite.

A major advantage of this apparatus is that it can be modular, with various parts being easily replaceable. For example, the catalyst may be provided in an easily-removable cartridge. Any associated heating elements may also be part of this cartridge.

In an especially preferred embodiment, the volatile liquid is provided in a replaceable cartridge that includes at least both the particulate filter, the liquid reservoir and the liquid disseminator. The invention therefore provides an apparatus adapted to both purify an air flow and disseminate a volatile liquid therein, comprising a source of volatile liquid, a disseminating element for the liquid and a catalyst adapted to remove pollutants at relatively low temperatures, the aforementioned elements being arranged such that the air flow passes first past the catalyst and then the disseminating element, the apparatus additionally comprising a filter for the removal of particulate matter, this filter being located such that the air flow encounters it before it encounters the catalyst, the filter along with the volatile liquid source and the disseminating element therefor comprising part of a single replaceable unit.

In another embodiment of the invention, the catalyst and any heating elements associated therewith form part of the abovementioned single replaceable unit. Such a unit is useful in cases in which the air flow is generated remotely, such as a ventilation system of a building or a motor vehicle. If necessary, means of providing electricity to heating elements associated with the catalyst may easily be provided.

The practical configuration of the apparatus hereinabove described is easily realisable by the skilled person. For example, if the air flow moves along a duct in a straight line from entrance to exit, be the duct part of a domestic apparatus or a ventilation duct, the duct may be supplied with suitable orifices through which the particulate filter and the disseminating element may be inserted. This is naturally not the only possible arrangement, and others can easily be envisaged. For example, in a domestic apparatus with an internal fan, it may be convenient that air be drawn in vertically and then blown horizontally by a fan. A single replaceable unit comprising particulate filter and disseminating element to suit this situation can easily be realised. The replaceable unit may be made of any suitable material, and it may be held in place by any convenient means, for example, screws, clips or snap fitting.

The apparatus provides in a single convenient package an air purifier and a disseminator of volatile liquid. The volatile liquid may be any desired volatile liquid, such as a fragrance, an insecticide, a medicament or a fungicide. The selection of the proportions of the apparatus and its component parts, the flow rate of the apparatus in relation to the size of atmosphere in which it is required to disseminate the vaporised liquid, the heating of the catalyst to the desired temperature and other parameters, are well within the skill of the art.

The invention is now further described with reference to the drawings, which depict preferred embodiments relating to domestic apparatus, but which are not to be construed as limiting in any way on the scope of the invention.

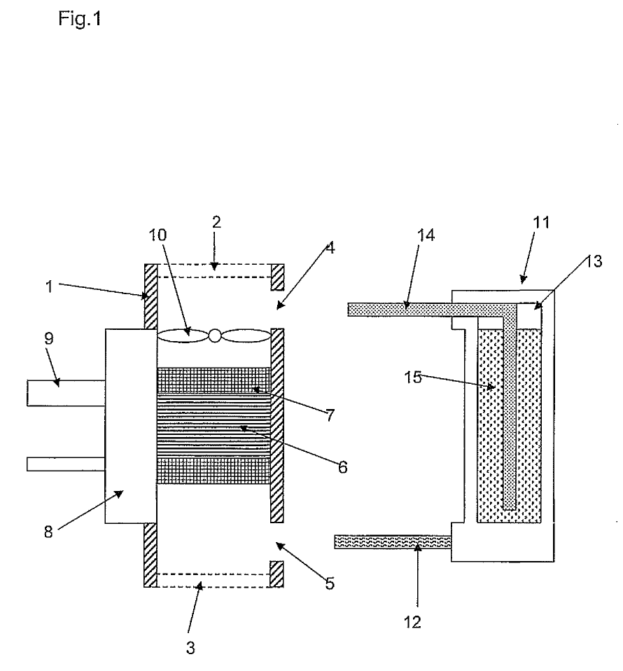

FIG. 1 depicts a schematic vertical cross-section through an embodiment, showing the two component parts of the apparatus separated.

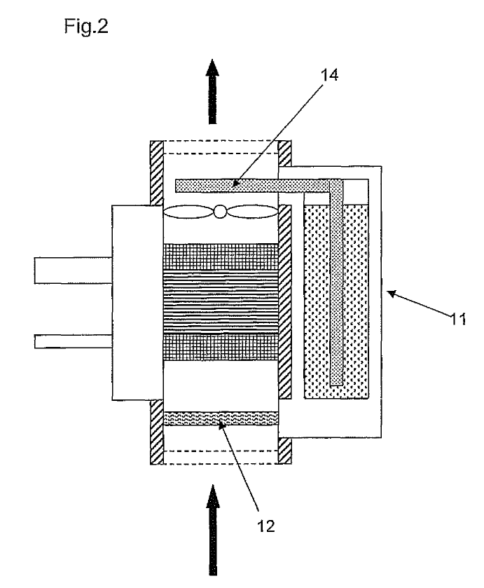

FIG. 2 depicts a schematic vertical cross-section through the apparatus of FIG. 1, showing the two parts making up the apparatus joined together ready for operation.

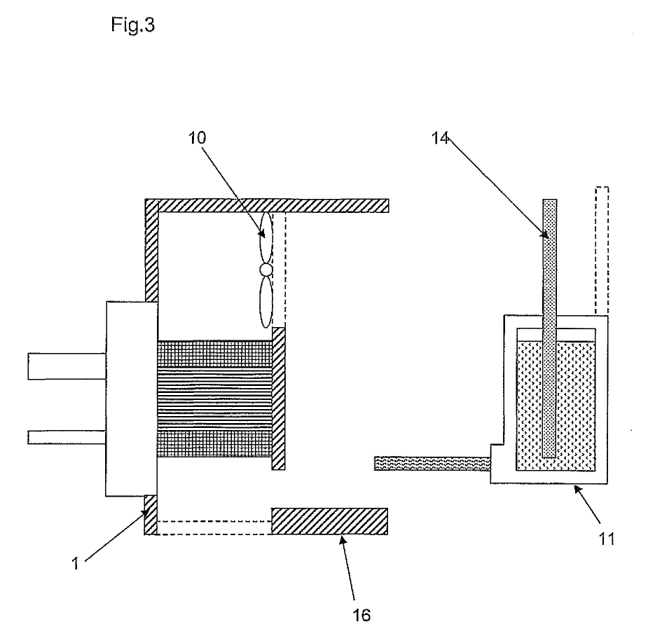

FIG. 3 depicts a schematic vertical cross-section through a second embodiment of the invention, showing, as in FIG. 1, the two component parts separated.

In all drawings, the dotted lines are representative of grilles that are fitted to the apparatus, to prevent the working parts being touched accidentally by fingers (such as those of children) or tools.

In FIG. 1, the apparatus comprises an essentially tubular outer casing 1 with a top vent 2 and bottom vent 3, which allow air flow in and out, and which in use is orientated such that the axis of the tube is vertical. Within the outer casing are located a fan 10 powered by mains electricity via power point pins 9 adapted to be plugged into a mains outlet and an associated transformer 8. The fan is arranged such that it draws air in through the bottom vent and expels it out through the top vent 2. Within the casing is also mounted a catalyst-containing monolith 6 surrounded by a heater element 7, this heater element also taking its power from the transformer 8.

On the reverse side of the casing from the power pins are located two orifices 4 and 5, 4 being positioned above the fan 10 and 5 below the catalyst monolith 6. These orifices are adapted to permit the insertion of, in the case of orifice 4, a volatile liquid disseminating element 14, and in the case of orifice 5, a fibrous filter 12. The filter 12 is dimensioned such that it spans completely the cross-sectional area of the casing, such that all air passing into the casing will have to pass through the filter. On the other hand, the disseminating element, which takes the form of a porous wick, does not span the entire cross-sectional area, but is in the form of an elongate rod, which extends into the diameter. The orifices 4 and 5 are dimensioned so that the filter and the element can be accommodated. In this embodiment, both disseminating element and filter extend essentially horizontally through the casing.

Both the filter and the element form part of a refill 12, which consists of a reservoir 13 containing volatile liquid I 1, the element extending from the upper part of the refill and the filter from the lower part. The wick 14 extends into the refill and then bends downwards through a right-angle to enter the reservoir and contact the liquid, such that it can draw liquid out of the reservoir to the horizontal part of the wick that extends into the casing. The refill is held in place on the casing by a plastics snap fitting arrangement (not shown).

For use, as shown in FIG. 2, a refill 11 is snapped into the casing. It can be seen that the wick 14 and the particulate filer 12 extend into the casing and into the air flow, when it commences. When the apparatus is plugged into a power point, both fan and heater start immediately to function, the fan drawing in air from below, as depicted by the lower thick black arrow, and sucking it through the filter and then the catalyst, which remove particulate and chemical impurities from the air. It then blows the air past the wick, and this entrains into the moving air the volatile liquid in vapour form, which is then blown into the atmosphere, as shown by the upper thick black arrow.

FIG. 3 depicts an alternative form of the apparatus of the invention, in which the fan 10 is mounted such that its axis of rotation is horizontal and it blows air horizontally. In this case, the wick 14 has no bend and is vertical when mounted in the apparatus. In this embodiment, the casing 1 additionally comprises a horizontal shelf 16, which extends therefrom away from the power pins, and on which the refill 11 rests. The air enters the casing vertically from underneath and is blown out horizontally.

The skilled person will readily be able to envisage many other variations of the apparatus of the invention, all of which are well within the skill of the art.

1. An apparatus adapted to both purify an air flow passing through it and disseminate a volatile liquid therein, comprising a source of volatile liquid, a disseminating element for the liquid and a catalyst adapted to remove pollutants at relatively low temperatures, the aforementioned elements being arranged such that the air flow encounters first the catalyst and then the disseminating element.

2. An apparatus according to claim 1, in which the air flow is provided by a fan.

3. An apparatus according to claim 2, in which the fan is placed such that it draws air over the catalyst and blows it over the disseminating element.

4. An apparatus according to claim 1, in which the catalyst comprises a precious metal and a metal oxide.

5. An apparatus according to claim 4, in which the precious metal is selected from platinum, palladium and gold, preferably gold.

6. An apparatus according to claim 4, in which the metal oxide is iron oxide.

7. An apparatus according to claim 4, in which the catalyst is supported on a metal monolith.

8. An apparatus according to claim 4, in which there is associated with the catalyst a heating element.

9. An apparatus according to claim 7, in which the monolith has a flexible printed resistor wrapped around it.

10. An apparatus according to claim 1, in which the apparatus additionally comprises a filter for the removal of particulate matter, this filter being located in the air flow before the catalyst, the filter along with the volatile liquid source and the disseminating element therefor comprising part of a single replaceable unit.

11. An apparatus according to claim 11, in which the catalyst and any heating element associated therewith forms part of the replaceable unit.

12. A method of simultaneously purifying and disseminating into an atmosphere a volatile liquid, comprising causing the atmosphere to pass in order over a catalyst adapted to remove pollutants at relatively low temperatures and a volatile liquid disseminating element.
